(19) United States
(12) Patent Application Publication
(10) Pub. No.: US 2023/0346281 A1
(43) Pub. Date: Nov. 2, 2023

(54) Fully Printed Electrode Biological Interface

(71) Applicant: Early Charm Ventures, Baltimore, MD (US)

(72) Inventor: Stephen Farias, Towson, MD (US)

(21) Appl. No.: 18/307,200

(22) Filed: Apr. 26, 2023

Related U.S. Application Data
(60) Provisional application No. 63/335,499, filed on Apr. 27, 2022.

(51) Int. Cl. A61B 5/25

(52) U.S. Cl. CPC A61B 5/25; A61B 2562/0209; A61B 2562/125

(57) ABSTRACT

Methods and apparatus are presented which allow for the in vitro measurement of cell viability in multilayered electronic biologic sensors. The use of printed layer composites containing conductive and nonconductive layers provide quantitative data on human tissue toxicology in real-time. The printing process permits a wide range of design options, like materials of composition, type of live cells, testing regimes, and live cell viability monitoring.

### CROSS-REFERENCE TO RELATED APPLICATIONS

This application claims the benefit of U.S. Provisional Application No. 63/335,499, filed Apr. 27, 2022 which is incorporated by reference herein in its entirety.

### TECHNICAL FIELD

Embodiments herein generally relate to printed in vitro assay devices. More specifically, one or more embodiments relate to preparing live cell impregnated or layered devices with real-time monitoring of cell viability.

### BACKGROUND

Toxicological testing via in vivo assays typically occurs toward the end of product development of a drug or consumer product, like in cosmetic, personal care, or pharma applications. At the end of the product development process, millions of dollars have already been spent on research and development. These in vivo screens are expensive, harmful to animals, and have the potential to kill products after years of effort and money have been expended. Ideally, an in vitro assay could be performed much earlier in the product development process which would avoid costly late-stage drug/product program terminations. To be effective, the in vitro assay should be rapid and inexpensive. The challenge is that current in vitro toxicological assays used for human tissue are slow, expensive, and unreliable. There is a need for an in vitro toxicology assay that provides quantitative data on human tissue, like those from cardiovascular, neurovascular, and brain samples, all in real-time.

### SUMMARY

Embodiments include a number of different methods for fabricating a multilayer electrode biologic sensor. One such method includes creating a design of the multilayer electrode biologic sensor and selecting a support matrix having a support matrix composition to deposit two or more composite layers. The method further includes depositing a first composite layer having a first composition on the support matrix. The method further includes applying a second composite layer having a second composition, at least partially, to overlie the first layer, such that the second layer at least partially bonds to the first composite layer. The method further includes applying a third composite layer having a third composition, at least partially, to overlie the second layer, such that the third layer at least partially bonds to the second composite layer. The method further includes applying one or more additional composite layers, having one or more additional compositions, at least partially, to overlie the previous layer, such that the one or more additional layers at least partially bonds to the previous composite layer, thereby to provide the design of the multilayer electrode biologic composite.

In certain embodiments, the support matrix is a sterilizable material. In certain embodiments, the sterilizable material is one of a polyester, a glass, a polyethylene, a silicone, a polycarbonate, or a combination thereof. In certain embodiments, the first composition is selected from one of a conductive ink, a biocompatible hydrogel, a live cell loaded biocompatible hydrogel, or live cells. In certain embodiments, the second composition is selected from one of a conductive ink, a biocompatible hydrogel, a live cell loaded biocompatible hydrogel, or live cells. In certain embodiments, the first composite layer and second composite layer are the same or different. In certain embodiments, the third composition is selected from one of a conductive ink, a biocompatible hydrogel, a live cell loaded biocompatible hydrogel, or live cells. In certain embodiments, the first composition, second composition, and third composition are the same or different. In certain embodiments, the one or more additional compositions are selected from one of a conductive ink, a biocompatible hydrogel, a live cell loaded biocompatible hydrogel, or live cells.

In certain embodiments, the first composition is a biocompatible hydrogel with a gel melt temperature of greater than about 40° C. In certain embodiments, the biocompatible hydrogel is one of a gelatin, a cellulosic, a polyethylene glycol, a poly-lysine, or a combination thereof. In certain embodiments, the first composition is a live cell loaded biocompatible hydrogel with a gel melt temperature of greater than about 40° C. In certain embodiments, the live cell biocompatible hydrogel is one of a gelatin, a cellulosic, a polyethylene glycol, a poly-lysine, or a combination thereof. In certain embodiments, the live cell loaded biocompatible hydrogel is loaded with between about 0.25 million cells per mL to about 2.50 million cells per mL. In certain embodiments, the first composition is a conductive ink with a resistivity of between about 10,000 mΩ·mm²/m to about 150,000 mΩ·mm²/m. In certain embodiments, the conductive ink comprises a carbon or a metallic particle retained in a biocompatible carrier matrix.

In certain embodiments, the second composition is a biocompatible hydrogel with a gel melt temperature of greater than about 40° C. In certain embodiments, the biocompatible hydrogel is one of a gelatin, a cellulosic, a polyethylene glycol, a poly-lysine, or a combination thereof. In certain embodiments, the second composition is a live cell loaded biocompatible hydrogel with a gel melt temperature of greater than about 40° C. In certain embodiments, the live cell biocompatible hydrogel is one of a gelatin, a cellulosic, a polyethylene glycol, a poly-lysine, or a combination thereof. In certain embodiments, the live cell loaded biocompatible hydrogel is loaded with between about 0.25 million cells per mL to about 2.50 million cells per mL. In certain embodiments, the second composition is a conductive ink with a resistivity of between about 10,000 mΩ·mm²/m to about 150,000 mΩ·mm²/m. In certain embodiments, the conductive ink comprises a carbon or a metallic particle retained in a biocompatible carrier matrix.

In certain embodiments, the third composition is a biocompatible hydrogel with a gel melt temperature of greater than about 40° C. In certain embodiments, the biocompatible hydrogel is one of a gelatin, a cellulosic, a polyethylene glycol, a poly-lysine, or a combination thereof. In certain embodiments, the second composition is a live cell loaded biocompatible hydrogel with a gel melt temperature of greater than about 40° C. In certain embodiments, the live cell biocompatible hydrogel is one of a gelatin, a cellulosic, a polyethylene glycol, a poly-lysine, or a combination thereof. In certain embodiments, the live cell loaded biocompatible hydrogel is loaded with between about 0.25 million cells per mL to about 2.50 million cells per mL. In certain embodiments, the third composition is a conductive ink with a resistivity of between about 10,000 mΩ·mm²/m to about 150,000 mΩ·mm²/m. In certain embodiments, the conductive ink comprises a carbon or a metallic particle retained in a biocompatible carrier matrix.

The method of claim 1, wherein the one or more additional compositions is selected from one of a conductive ink, a biocompatible hydrogel, a live cell loaded biocompatible hydrogel, or live cells. In certain embodiments, the additional composition is a biocompatible hydrogel with a gel melt temperature of greater than about 40° C. In certain embodiments, the biocompatible hydrogel is one of a gelatin, a cellulosic, a polyethylene glycol, a poly-lysine, or a combination thereof. In certain embodiments, the additional composition is a live cell loaded biocompatible hydrogel with a gel melt temperature of greater than about 40° C. In certain embodiments, the live cell biocompatible hydrogel is one of a gelatin, a cellulosic, a polyethylene glycol, a poly-lysine, or a combination thereof. In certain embodiments, the live cell loaded biocompatible hydrogel is loaded with between about 0.25 million cells per mL to about 2.50 million cells per mL. In certain embodiments, the additional composition is a conductive ink with a resistivity of between about 10,000 mΩ·mm²/m to about 150,000 mΩ·mm²/m. In certain embodiments, the conductive ink comprises a carbon or a metallic particle retained in a biocompatible carrier matrix.

In another embodiment, an apparatus for toxicity screening in live cells is disclosed. One such apparatus includes a multilayer biologic apparatus for toxicity screening in live cells comprising a sterilizable support matrix, a tissue layer, and a conductive layer. In certain embodiments, the sterilizable support matrix is a one of a polyester, a glass, a polyethylene, a silicone, a polycarbonate, or a combination thereof. In certain embodiments, the tissue layer is a biocompatible hydrogel impregnated with live cells or a live cell layer. In certain embodiments, the biocompatible hydrogel is one of a gelatin, a cellulosic, a polyethylene glycol, a poly-lysine, or a combination thereof with a gel melt(set) temperature of greater than about 40° C.

In additional embodiments, a method for determining the viability of living cells under varying environments is disclosed. One such method includes preparing a multilayer electrode biologic sensor with a live cell layer, incubating the live cell layer under a range of environments, introducing test compounds to the live cell layer, monitoring an electrical cellular impedance spectroscopy of the live cell layer, and evaluating a live cell viability from the electrical cellular impedance spectroscopy. In certain embodiments, the live cell layer comprises mammalian cells. In certain embodiments, the method further comprises sampling the live cell layer for further evaluation.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. According to common practice, the various features of the drawings discussed below are not necessarily drawn to scale. Dimensions of various features and elements in the drawings may be expanded or reduced to more clearly illustrate embodiments of the disclosure.

Embodiments will be readily understood by the following detailed description in conjunction with the accompanying drawings. To facilitate this description, like reference numerals designate like structural elements or procedures in a method. Embodiments are illustrated by way of example and not by way of limitation in the figures of the accompanying drawings.

FIGS. 1A and 1B schematically illustrate a multilayer biologic electrode sensor, in accordance with various embodiments.

FIG. 2 schematically illustrates a stacked multilayered electrode biologic sensor configured to record resistivity values of the live cell layer, in accordance with various embodiments.

### DETAILED DESCRIPTION

The present disclosure describes various embodiments related to methods, apparatus, and systems for determining the toxicity of environmental conditions and toxicological agents on mammalian live cells. In various embodiments, the apparatus may be implemented to monitor cellular health, toxicology response, ischemia, and apoptosis. In certain embodiments, real-time monitoring of live cells is achieved through Electric Cellular-Substrate Impedance Sensing (ECIS) using printed electrodes embedded below, above, or within the live cell layers of the apparatus. In certain embodiments, the frequency of the ECIS monitoring will be a single frequency, a frequency range, or a full spectrum sweep of frequencies to monitor different cellular kinetics associated with environment or toxicological agents.

In certain embodiments, the method of fabrication includes creating a design of an electrode biologic sensor containing two or more layers deposited on a substrate layer. In certain embodiments, the layers are biocompatible hydrogels, live cells, or conductive inks. In certain embodiments, the deposited layers are stacked, side by side, or a combination thereof.

In additional embodiments, a method for determining the viability of living cells under varying environments is disclosed. One such method includes preparing a multilayer electrode biologic sensor with a live cell layer, incubating the live cell layer under a range of environments, introducing test compounds to the live cell layer, monitoring an electrical cellular impedance spectroscopy of the live cell layer, and evaluating a live cell viability from the electrical cellular impedance spectroscopy.

In the following description, numerous details are set forth in order to provide a thorough understanding of the various embodiments. In other instances, well-known processes, devices, and systems may not been described in particular detail in order not to unnecessarily obscure the various embodiments. Additionally, illustrations of the various embodiments may omit certain features or details in order to not obscure the various embodiments.

In the following detailed description, reference is made to the accompanying drawings that form a part of this disclosure. Like numerals may designate like parts throughout the drawings. The drawings may provide an illustration of some of the various embodiments in which the subject matter of the present disclosure may be practiced. Other embodiments may be utilized, and logical changes may be made without departing from the scope of this disclosure.

The description may use the phrases “in some embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous.

The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous. The term “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “removing,” “removed,” “reducing,” “reduced,” or any variation thereof, when used in the claims and/or the specification includes any measurable decrease of one or more components in a mixture to achieve a desired result. The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.” The term “plurality” as used herein refers to two or more items or components. The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

Various embodiments disclosed and described may relate to the viability of live cells under various environments. The apparatus provides a scalable in vitro toxicity screening system via a novel screen printed ECIS technology. In certain embodiments, the apparatus provides real-time quantitative data on the effects of environmental conditions or toxicological agents on a variety of human cells. In certain embodiments, the printed live cells are mammalian, such as endothelial, gastrointestinal (GI), optic nerve, brain, cancer cells, or combinations thereof. In certain embodiments, the live cell layer is between about 0.25 million cells/mL to about 2.50 million cells/mL. In certain embodiments, the live cell layer is printed above or below the adjacent layer. In certain embodiments, the live cells are printed within a biologically compatible hydrogel. In certain embodiments, live cell layers of different composition can be stacked, either as an independent layer or as a mixed layer to test viability against specific environmental conditions.

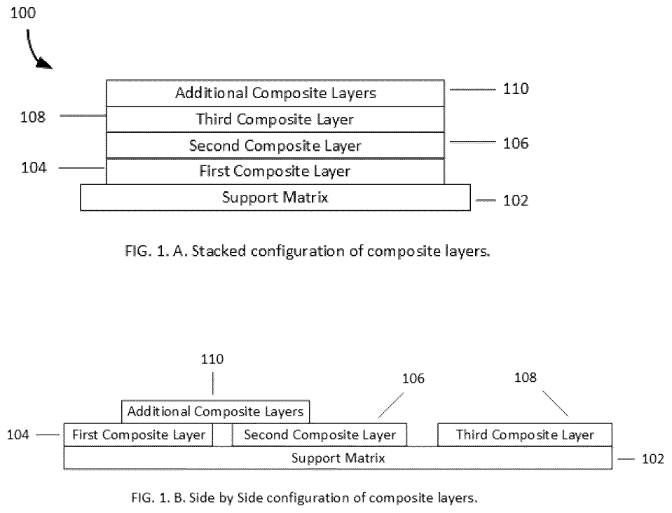

In certain embodiments, the layers are deposited using piston, screw, or pneumatic extrusion-based bioprinting. In certain embodiments, the layers are deposited using screen printing in which the material is passed through a patterned screen. Each pass creates a unique deposition layer consistent with the screen pattern. In certain embodiments, the deposition of layers use the same or different screen pattern. In certain embodiments, the deposition of layers can be stacked on top of each other or offset as depicted in FIG. 1. In certain embodiments, the screen-printed layers are embedded with electronic sensors for impedance readouts that correspond to cellular response to potential toxicological agents.

In certain embodiments, the electronic sensors are used to measure the resistivity of the live cell layer. Electrical impedance measurements have advantages compared to trans endothelial electrical resistance (TEER) or other measures of electrical resistance. The power of electrical impendence, and specifically ECIS, is that it can detect changes in live cell resistivity across a wide frequency range. In certain embodiments, screen printing patterns of tissue and electrodes can be matched repeatedly to the same precise geometry. In certain embodiments, screen printing in layers also allows for electrodes to interface on the top, bottom, and/or middle of the tissue layer at discreet and precise locations.

In certain embodiments, quantitative real-time electrical response measurements of the live cell layer are taken as the live cell layer is exposed to various toxins. Traditional chemical toxicity and molecular studies, e.g. fluorescence microscopy, require tissue sampling and secondary analysis done after the change in environment and not in real-time.

In certain embodiments, a biocompatible hydrogel layer is used to embed, incorporate, or support the live cell layer. In certain embodiments, the melt temperature of the biocompatible hydrogel layer is greater than 40° C. making it suitable to print as a 10% solution. Once the gel cools to room temperature, the gelatin becomes a manipulatable hydrogel meaning that it holds its shape immediately after printing. In certain embodiments, the biocompatible hydrogel is a naturally derived gelatin, collagen, sodium alginate, or basement-membrane matrix referred to as Matrigel. In certain embodiments, the biocompatible hydrogel is a synthetically derived polyethylene glycol (PEG), poly-lysine (PL), or combination thereof. In certain embodiments, the biocompatible hydrogel is a PEG:PL mixture in a ratio of between about 1:10 to about 1:20 by weight. In certain embodiments, the biocompatible hydrogel is a PEG:PL mixture in a ratio of between about 1:1 to about 1:50 by weight.

In certain embodiments, prior to application the hydrogel is filtered through a filter with a pore size of about 0.45 μm. In certain embodiments, each non live cell layer is exposed to UV light for sterilization during solution preparation, after printing as a composite layer, or both. In certain embodiments, each composite layer of the multilayer electrode biologic sensor is deposited in a thickness of between about 5 μm to about 5 mm. In certain embodiments, each layer may have the same or different thicknesses depending on the nature of the live cells and real-time measurement requirements.

In certain embodiments, conductive inks are used to print a screen pattern in the form of an electrode. In certain embodiments the conductive ink contains carbon or metallic particle. In certain embodiments, the conductive ink has a resistivity of about 20,000 mΩ/sq/mi to about 100,000 mΩ/sq/mi. In certain embodiments, the conductive ink has a viscosity of about 15 Pa·s to about 85 Pa·s. In certain embodiments, the conductive ink has a resistivity of about 10,000 mΩ/sq/mi to about 150,000 mΩ/sq/mi. In certain embodiments, the electrode design is a broken outer semi-circle and a solid inner full circle. In certain embodiments, the electrode design provides for two poles enabling the measurable flow of electrons when a substrate is placed to bridge the two poles.

In certain embodiments, the patterned screen mesh size is between about 100 μm to about 400 μm. The print screen mesh size affects the resolution of the printed layers. In certain embodiments, the patterned screen mesh size is between about 150 μm to about 177 μm. In certain embodiments, the electrode layer screen mesh size is between about 120 μm to about 150 μm.

In certain embodiments, additional components, like drugs, toxins, growth accelerators, therapeutic compounds, and other agents which affect the live cells of interest, can be added to composite layers adjacent to the live cell layers. In certain embodiments, the diffusion rates of the additional components from the adjacent composite layers can be tailored to provide a controlled or slow release of components into the live cell layer.

FIG. 1.A. is a schematic illustration of a support matrix 102 upon which a first composite layer 104 is deposited. A second composite layer 106 is applied to the first composite layer 104 such that second composite layer 106 overlays first composite layer 104, at least partially, and partially bonds to the first composite layer 104. A third composite layer 108 is applied to the second composite layer 106 such that third composite layer 108 overlays second composite layer 106, at least partially, and partially bonds to the second composite layer 106. One or more additional composite layers 110 are applied to overlie the previous layer, at least partially, such that the one or more additional layers at least partially bonds to the previous composite layer, thereby to provide the design of the stacked multilayer electrode biologic composite. In certain embodiments, the applied layers can be the same or of different composition to provide the design of the stacked multilayer electrode biologic composite. In certain embodiments, the applied layers can be the same or of different thickness to provide the design of the stacked multilayer electrode biologic composite. In certain embodiments, the deposited and applied layers are unbonded to the previous layer.

FIG. 1.B. is a schematic illustration of a support matrix 102 upon which a first composite layer 104 is deposited to a portion of the support matrix 102. A second composite layer 106 is deposited to a different portion of the support matrix 102. A third composite layer 108 is deposited to yet another portion of the support matrix such that the first composite layer 104, second composite layer 106, and third composite layer 108 are in a side-by-side configuration. One or more additional composite layers 110 are applied to overlie the first composite layer 104 and the second composite layer 106, at least partially, such that the one or more additional layers 110 at least partially bonds to the first composite layer 104 and second composite layer 106. In certain embodiments, the side-by-side configuration of the first composite layer, the second composite layer, and the third composite result in a multichannel, multi-well, or high throughput screening array. In certain embodiments, the one or more composite layers 110 can be applied to each deposited composite layer independently or simultaneously. In certain embodiments, the first composite layer 104, the second composite layer 106, and the third composite layer 108 can be of the same or different compositions. In certain embodiments, the deposited and applied layers can be the same or of different thickness. In certain embodiments, the deposited and applied layers are unbonded to the previous layer.

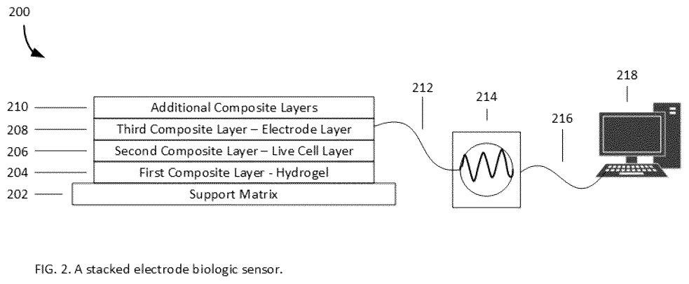

FIG. 2 is a schematic illustration of an apparatus to measure the vitality of live cells under various conditions using ECIS. A first composite layer 204 consisting of a hydrogel is deposited on a support matrix 202. A second composite layer 206, consisting of live cells is applied to the first composite layer 204. A third composite layer 208 consisting of an electrode layer is applied to the second composite layer. One or more additional layers 210 can be applied as needed to provide the design of the apparatus. Impedance values of the live cell layer 206 as measured by the electrode layer 208 are transmitted 212 to a receiver 214, and the digital values are transmitted 216 to a controller 218 for numerical computation, analysis, and graphical display. In certain embodiments, the electrode layer is a printed layer using conductive inks with the printed electrode configured as parallel lines, concentric broken and unbroken circles, blocks, and tapered lines. In certain embodiments, the electrode consists of two or more poles connected to a receiver. In certain embodiments, the receiver can be one or more devices like an oscilloscope.

In certain embodiments, the controller may be part of a control system to manage environmental conditions, stimuli, and data collection of the electrode biologic interface. In an example, the control system may include one or more controllers. In certain embodiments, the controller may be in signal communication with various other controllers. In certain embodiments, the controller may be considered a supervisory controller.

In certain embodiments, each controller may include a machine-readable storage medium and one or more processors. In certain embodiments, a “machine readable storage medium” may be any electronic, magnetic, optical, or other physical storage apparatus to contain or store information such as executable instructions, data, and the like. For example, any machine-readable storage medium described herein may be any of random-access memory (RAM), volatile memory, non-volatile memory, flash memory, a storage drive (e.g., hard drive), a solid state drive, and type of storage disc, and the like, or a combination thereof.

In certain embodiments, the machine readable-readable storage medium may store or include instructions executable by a processor. As used herein, a “processor” may include, for example, one or more processors included in a single device or spread across multiple computing devices. In certain embodiments, the processor may be at least one of a central processing unit (CPU), a semiconductor-based microprocessor, a graphics processing unit (GPU), a field-programmable gate array (FPGA) to retrieve and execute instructions, a real-time processor (RTP), other electronic circuitry suitable for the retrieval and execution of instruction stored on a machine-readable storage medium, or a combination thereof.

In certain embodiments, “signal communication” includes electrical communication such as hard wiring two components together or wireless communication, as understood by those in the art. In certain embodiments, the wireless communication may be Wi-Fi®, Bluetooth®, Zigbee, or forms of near field communications. In certain embodiments, signal communication may include one or more intermediate controllers or relays disposed between elements that are in signal communication with one another.

In certain embodiments, the controller may receive commands from an operator, from a predetermined sequence of steps embedded in code language, or from a self-generated set of commands from analysis of conditions with the electrode biological interface. In certain embodiments, the self-generated commands may come from analysis of datasets using algorithms and statistical models to make predictions such as neural net learning systems, or artificial intelligence (AI).

### EXAMPLES

Other objects, features and advantages of the disclosure will become apparent from the foregoing figures, detailed description, and examples. It should be understood, however, that the figures, detailed description, and examples, while indicating specific embodiments of the disclosure, are given by way of illustration only and are not meant to be limiting. Additionally, it is contemplated that changes and modifications within the spirit and scope of the disclosure will become apparent to those skilled in the art from the detailed description. In further embodiments, features from specific embodiments may be combined with features from other embodiments. For example, features from one embodiment may be combined with features from any of the other embodiments. In further embodiments, additional features may be added to the specific embodiments described herein.

### Example 1

In Example 1, an experimental method is disclosed and described for preparing a printed multilayer electrode biologic sensor including embodiments of the multilayer electrode biologic sensor disclosed and claimed.

A multilayer electrode biologic sensor was prepared by using a Polyethylene Glycol (PEG) and poly-L-Lysine hydrogel in a ratio 1:10 and 1:20 by weight and hydrated with deionized water to a viscosity compatible with the selected screen mesh size. The biocompatible hydrogels were passed through a patterned mesh to deposit a pattern on a substrate layer. The biocompatible hydrogel was selected to set within about ten to about 30 minutes of application. After the set period, a solution of live cells, 500,000 cells per mL of human hepatocellular carcinoma HepG2 cells [HepG2], was passed through the patterned mesh to deposit a layer of live cells on the biocompatible hydrogel. A conductive ink was passed through a patterned mesh corresponding to the electrode pattern to deposit an electrode layer on the live cell layer. The resulting composite was incubated at 37° C. for 72 hours. At intervals of 1 hour, 24 hours, and 72 hours live cell viability was assessed using Calcein AM (as provided by ThermoFisher) and a plate reader operating between 494 nm and 517 nm, Table 1.

TABLE 1
Live Cell Viability

| | Cell Viability, % | | |
|---|---|---|---|
| | 1 Hour | 24 hours | 72 Hours |
| Control-Media Growth | 100 | 79 | 70 |
| Only | | | |
| 1:10 PEG/PLL Hydrogel, | 84 | 41 | 25 |
| Live Cell Layered | | | |
| 1:10 PEG/PLL Hydrogel, | 55 | 23 | 20 |
| Impregnated | | | |
| 1:20 PEG/PLL Hydrogel, | 59 | 45 | 40 |
| Live Cell Layered | | | |
| 1:20 PEG/PLL Hydrogel, | 58 | 25 | 21 |
| Impregnated | | | |

### Example 2

In Example 2, an experimental method is disclosed and described for using a printed multilayer electrode biologic sensor to determine the viability of live cells including embodiments of the multilayer electrode biologic sensor disclosed and claimed.

A multilayer electrode biologic sensor was prepared according to Example 1 and containing layered live cells at concentrations of 500,000 cells/mL and 2,000,000 cells/mL. At various sampling intervals, 1 hour and 48 hours, cell electrochemical impedance spectrometry (CEIS) was recorded and compared to Calcein AM live cell data, Table 2. A constant small alternating current is applied between the electrodes and the potential across is measured. The insulating properties of the cell membrane create a resistance towards the electrical current flow resulting in an increased electrical potential between the electrodes which is directly related to the viability of live cells. Measuring cellular impedance in this manner allows the automated study of cell attachment, growth, morphology, function, and motility. A reduction in impedance amplitude at the higher frequencies (>100 Hz) as well as an increase in the cell viability is observed when depositing HepG2 cells at higher densities. In both conditions (low and high cellular density), viability significantly reduced within 48 hours of cell deposition.

TABLE 2
Live Cell Viability As Measured By CEIS.

| | Cell Viability, % | |
|---|---|---|
| | 1 Hour | 48 hours |
| Control Hydrogel-Gelatin | 100 | 98 |
| 1:10 PEG/PLL Hydrogel, Live Cell | 30 | 5 |
| Layered, 500,000 cells/mL | | |
| 1:10 PEG/PLL Hydrogel, Live Cell | 42 | 4 |
| Layered, 2,000,000 cells/mL | | |

A toxicological agent, Ethanol (EtOH) was used as a clinically relevant cytotoxic agent for the HepG2 hepatocytes tissue layers, HEPG2 (2 million cells/mL) with PEG-PLL (1:10) hydrogel. CEIS recordings were obtained at 1 hour and 24 hours post deposition with and without the cytoxic agent, Table 3. A final CEIS recording was obtained from these cells following a 12 hour exposure to EtOH (70% final). A marked increase in amplitude with additional recovery time post deposition (1 vs 24 hours). Introduction of cytotoxic EtOH (12 hours) also increased the observed amplitude of CEIS recordings.

TABLE 3
Live cell viability in the presence of a cytotoxic agent.

| | Cell Viability, % | |
|---|---|---|
| | 1 Hour | 48 hours |
| Control Hydrogel-Gelatin | 100 | 98 |
| 1:10 PEG/PLL Hydrogel, Live Cell | 30 | 5 |
| Layered, 500,000 cells/mL | | |
| 1:10 PEG/PLL Hydrogel, Live Cell | 42 | 4 |
| Layered, 2,000,000 cells/mL | | |

What is claimed is:

1. A method for fabricating a multilayer electrode biologic sensor, the method comprising:
creating a design of the multilayer electrode biologic sensor;
selecting a support matrix having a support matrix composition to deposit two or more composite layers;
depositing a first composite layer having a first composition on the support matrix;
applying a second composite layer having a second composition, at least partially, to overlie the first layer, such that the second layer at least partially bonds to the first composite layer;
applying a third composite layer having a third composition, at least partially, to overlie the second layer, such that the third layer at least partially bonds to the second composite layer; and
applying one or more additional composite layers, having one or more additional compositions, at least partially, to overlie the previous layer, such that the one or more additional layers at least partially bonds to the previous composite layer, thereby to provide the design of the multilayer electrode biologic composite.

2. The method of claim 1, wherein the support matrix is a sterilizable material.

3. The method of claim 2, wherein the sterilizable material is one of a polyester, a glass, a polyethylene, a silicone, a polycarbonate, or a combination thereof.

4. The method of claim 1, wherein the first composition is selected from one of a conductive ink, a biocompatible hydrogel, a live cell loaded biocompatible hydrogel, or live cells.

5. The method of claim 1, wherein the first composition is a biocompatible hydrogel with a gel melt(set) temperature of greater than about 40° C.

6. The method of claim 5, wherein the biocompatible hydrogel is one of a gelatin, a cellulosic, a polyethylene glycol, a poly-lysine, or a combination thereof.

7. The method of claim 1, wherein the first composition is a live cell loaded biocompatible hydrogel with a gel melt(set) temperature of greater than about 40° C.

8. The method of claim 6, wherein the live cell loaded biocompatible hydrogel is one of a gelatin, a cellulosic, a polyethylene glycol, a poly-lysine, or a combination thereof.

9. The method of claim 6, wherein the live cell loaded biocompatible hydrogel is loaded with between about 0.25 million cells per mL to about 2.50 million cells per mL.

10. The method of claim 1, wherein the first composition is a conductive ink with a resistivity of between about 10,000 mΩ·mm²/m to about 150,000 mΩ·mm²/m.

11. The method of claim 10, wherein the conductive ink comprises a carbon or a metallic particle retained in a biocompatible carrier matrix.

12. The method of claim 1, wherein the second composition is selected from one of a conductive ink, a biocompatible hydrogel, a live cell loaded biocompatible hydrogel, or live cells.

13. The method of claim 1, wherein the third composition comprises is selected from one of a conductive ink, a biocompatible hydrogel, a live cell loaded biocompatible hydrogel, or live cells.

14. The method of claim 1, wherein the one or more additional compositions is selected from one of a conductive ink, a biocompatible hydrogel, a live cell loaded biocompatible hydrogel, or live cells.

15. A multilayer biologic apparatus for toxicity screening in live cells, the apparatus comprising:
a sterilizable support matrix;
a tissue layer; and
a conductive layer.

16. The apparatus of claim 14, wherein the sterilizable support matrix is a one of a polyester, a glass, a polyethylene, a silicone, a polycarbonate, or a combination thereof.

17. The apparatus of claim 14, wherein the tissue layer comprises a biocompatible hydrogel impregnated with live cells or a live cell layer.

18. The apparatus of claim 16, wherein the biocompatible hydrogel is one of a gelatin, a cellulosic, a polyethylene glycol, a poly-lysine, or a combination thereof with a gel melt(set) temperature of greater than about 40° C.

19. A method for determining the viability of living cells under varying environments, the method comprising:
preparing a multilayer electrode biologic sensor with a live cell layer;
incubating the live cell layer under a range of environments;
introducing test compounds to the live cell layer;
monitoring an electrical cellular impedance spectroscopy of the live cell layer; and
evaluating a live cell viability from the electrical cellular impedance spectroscopy.

20. The method of claim 18, wherein the method further comprises sampling the live cell layer for further evaluation.
